(19) United States
(12) Patent Application Publication
(10) Pub. No.: US 2009/0317294 A1
(43) Pub. Date: Dec. 24, 2009

(54) Method of low-temperature dry sterilization and apparatus therefor

(76) Inventors: Takehiko Sato, Miyagi (JP); Shuitsu Fujii, Hiroshima (JP); Takuya Urayama, Hiroshima (JP); Akinori Shida, Hiroshima (JP); Raju Ramasamy, Hiroshima (JP); Kazunari Fujioka, Hiroshima (JP); Tatsuyuki Nakatani, Hiroshima (JP)

Correspondence Address: Kirschstein, Israel, Schiffmiller & Pieroni, P.C., 425 FIFTH AVENUE, 5TH FLOOR, NEW YORK, NY, 10016-2223, US

(21) Appl. No.: 11/990,975

(22) PCT Filed: Aug. 25, 2005
(86) PCT No.: PCT/JP2005/015431

§ 371 Date: Jul. 15, 2009

(51) Int. Cl. A61L 2/02; A61L 2/00

(52) U.S. Cl. 422/29; 422/292

(57) ABSTRACT

A method of low temperature sterilization that with the use of a compact simple apparatus, is capable of safe, unfailing sterilization. There is provided an apparatus comprising gas cylinder (gas supply source)(8), high energy particle generation part (1-3) capable of generating a gas of temperature nonequilibrium condition containing high energy particles through exciting of the gas supplied from the gas cylinder (8) and gas blow part (4) capable of jetting the gas of temperature nonequilibrium condition generated by the high energy particle generation part (1-3) over pathogenic microbes positioned outside.

### TECHNICAL FIELD

The present invention relates to a technique for killing bacteria living on the surface of objects or living organisms by use of high energy particles or the like in the air.

### TECHNICAL BACKGROUND

Recently, infectious diseases caused by bacteria such as severe acute respiratory syndrome (SARS) or avian flu suddenly and globally occur and cause a serious social problem. People are highly aware of this problem and ask for a safe and easy-to-handle sterilization and disinfection method.

Meanwhile, in a medical institution or a general household, sterilization and disinfection is generally carried out by use of antiseptic solution. However, there is no perfect antiseptic solution which combines safety and effectiveness and effective and therefore, different types of antiseptic solutions are only used in accordance with intended use. Sterilization and disinfection in a breeding area of farm animals for food currently relies on dispersion of disinfectant and is not sufficient. Therefore, development of prevention transmission technique for the farm animals is also a pressing problem.

Furthermore, a disinfection device using ethylene oxide gas, a disinfection device using a hydrogen peroxide low-temperature gas plasma and a disinfection device using radiation or the like are known as conventional compact low-temperature disinfection devices for medical use. However, the disinfection device using ethylene oxide gas has a disadvantage of using carcinogenic material which is legal restrained. The disinfection device using hydrogen peroxide low-temperature gas plasma requires a vacuum device, is high in cost, and operation of the apparatus is not easy. The disinfection device using radiation requires an expensive radiation generator and the limited installation location. Moreover, each of the disinfection devices are intended to sterilize medical equipments and batch sterilization method is adopted. Therefore, an object of sterilization is limited.
[Patent Document 1 ] Japanese Laid-Open Patent Publication No. H6-23023
[Patent Document 2] Japanese Laid-Open Patent Publication No 2002-85531

### DISCLOSURE OF THE INVENTION

### Problem to be Solved by the Invention

It is an object of the present invention to provide a low-temperature sterilization method achieving safe and reliable sterilization by use of a compact and simple apparatus.

### Means for Solving the Problem

To solve the above-mentioned problem, according to the present invention, there is provided a dry sterilization method comprising the steps of: generating gas of temperature nonequilibrium condition containing high-energy particles by exciting gas; and spraying pathogenic microorganisms with the gas of temperature nonequilibrium condition so as to kill the pathogenic microorganisms.

Here, “the gas of temperature nonequilibrium condition” means, for example, gas which contains particles having internal energy high enough to kill pathogenic microorganisms while, on the other hand, has small thermal energy and an energy condition suitable for accomplish the end desired.

To solve the above-mentioned problem, according to the present invention, there is provided a low-temperature dry disinfection device comprising: a gas supply source; a high-energy particle generator for generating gas of temperature nonequilibrium condition containing high-energy particles by exciting the gas supplied from the gas supply source; and a gas spraying unit for spraying external pathogenic microorganisms with the gas of temperature nonequilibrium condition generated by the high-energy particle generator.

In the above-mentioned configuration, it is preferable that the high-energy particle generator further comprises: a chamber for receiving gas supplied from the gas supply source; an electromagnetic field generation unit for providing the chamber with an electromagnetic field for exciting the gas in the chamber; and a high-voltage power supply for supplying power voltage to the electromagnetic field generation unit, the gas spraying unit further comprising a gas spraying pipe connected to the chamber.

Furthermore, it is preferable that the high-energy particle generator further comprises a cooling apparatus for cooling down the gas of temperature nonequilibrium condition before the gas is introduced into the gas spraying unit. It is also preferable that the gas supply source supplies a single type of gas or a mixed gas of more than two types of gases.

Furthermore, it is preferable that the high-energy particle generator further comprises a flow rate regulating valve arranged at a gas supply port for receiving gas from the gas supply source. It is also preferable that at least one of the high-energy particle generator and the gas spraying unit is provided with means for mixing steam into the gas of temperature nonequilibrium condition.

### Effect Of The Invention

According to the present invention, sterilization is carried out by spraying an object or living organisms with gas of temperature nonequilibrium condition including high energy particles, thereby sterilization effect enough to kill bacteria can be obtained, while damage to the object or the living creature can be significantly reduced. In addition, when metals or other heat resistant materials are selected as an object to be sterilized, by injecting the gas at the temperature of more than 50° C., it is possible to cut down the time for sterilization.

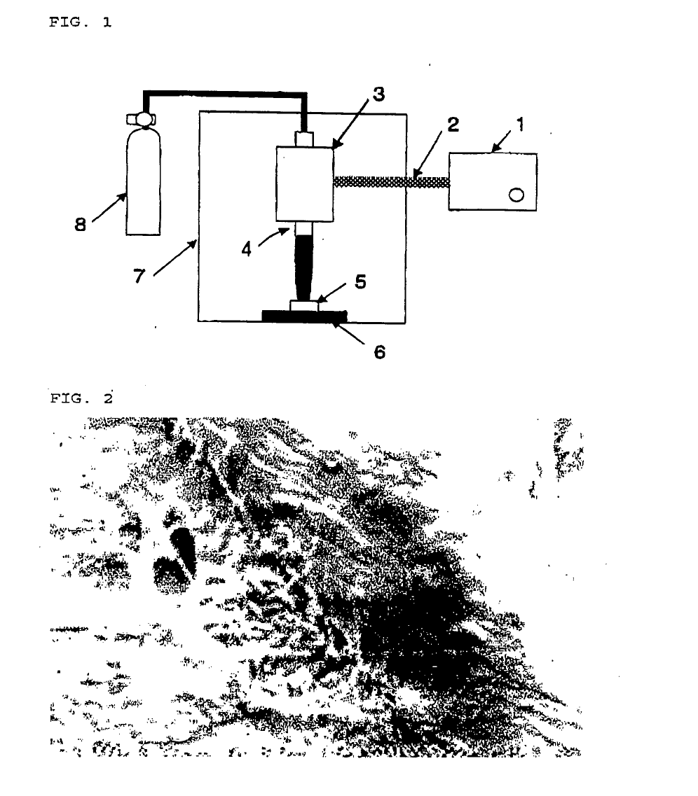

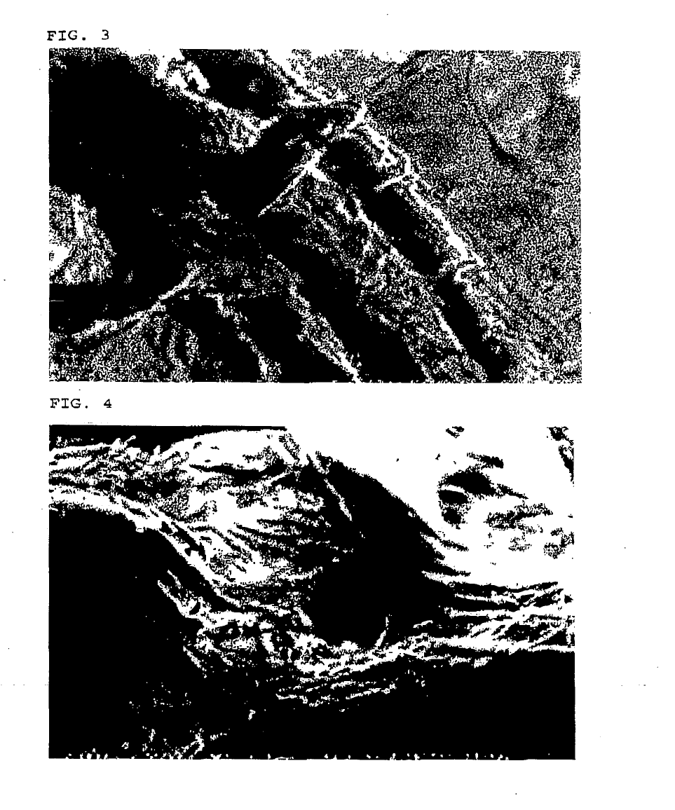

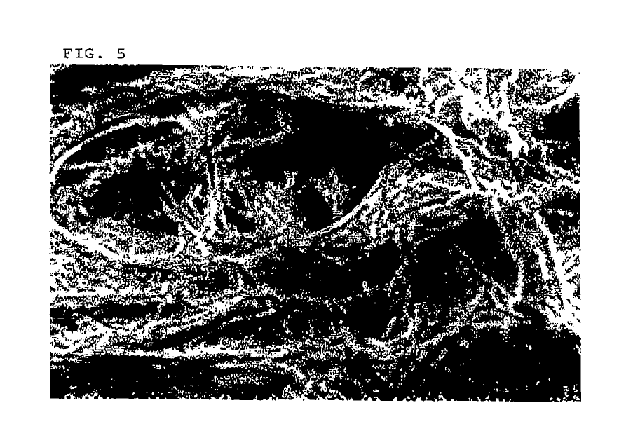

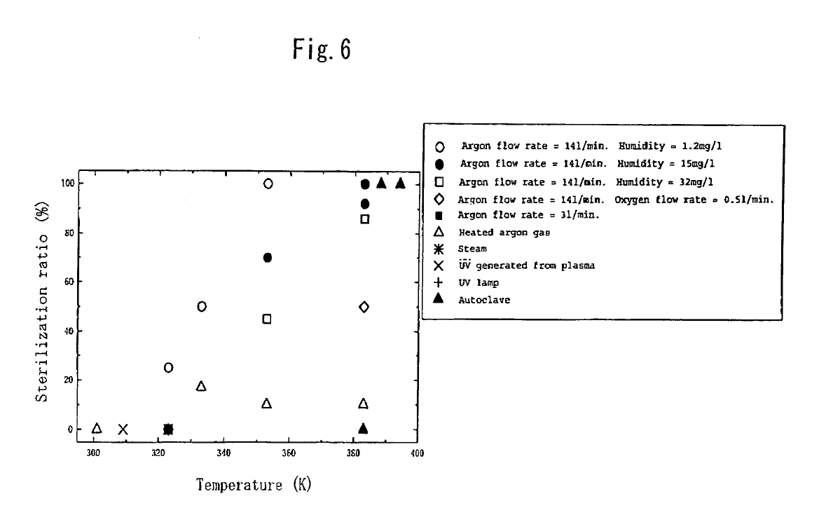

### BRIEF DESCRIPTION OF THE DRAWINGS

[FIG. 1] A view showing a schematic configuration of a low temperature dry disinfection device according to one embodiment of the present invention.

[FIG. 2] A photograph of spores of Bacillus subtilis coated on a surface of a sample which is not sterilized.

[FIG. 3] A photograph of spores of Bacillus subtilis coated on a surface of a sample which is sterilized by argon plasma radiation (353K).

[FIG. 4] A photograph of spores of Bacillus subtilis coated on a surface of a sample which is sterilized by heated argon gas (353K).

[FIG. 5] A photograph of spores of Bacillus subtilis coated on a surface of a sample which is sterilized by UV radiation.

[FIG. 6] A graph showing difference in sterilization ratio by sterilization temperature and sterilization methods.

### DESCRIPTION OF THE REFERENCE NUMERALS

1. Microwave power source
2. Co-axial cable
3. Plasma torch
4. Gas blow part
5. Sample
6. Substrate
7. Draft chamber
8. Gas cylinder

### THE BEST MODE FOR CARRYING OUT THE INVENTION

Hereafter, a preferred embodiment of the present invention will be explained with reference to attached drawings. FIG. 1 is a view showing a schematic configuration of a low temperature dry disinfection device according to one embodiment of the present invention.

According to FIG. 1, the low temperature dry disinfection device of the present invention includes a gas cylinder (gas supply source) 8, high energy particle generation parts 1-3 for exciting gas supplied from the gas cylinder 8 so as to generate gas of temperature nonequilibrium condition including high energy particles, and a gas injection part for spraying external pathogenic organisms with the gas of temperature nonequilibrium condition generated by the high energy particle generation parts 1-3.

In the present embodiment, a single gas cylinder 8 is provided and a single type of gas is supplied. However, a mixed gas of more than two types of gases maybe supplied from different gas cylinders.

The high energy particle generation parts 1-3 include, according to this embodiment, a microwave plasma source. The microwave plasma source includes a plasma torch 3, a microwave power source 1, and a co-axial cable 2 for supplying power from the microwave power source 1 to the plasma torch 3. Although not shown, the plasma torch 3 includes a chamber for receiving the gas supplied from the gas cylinder 8 and an electromagnetic field generator for providing the chamber with an electromagnetic field to excite gas in the chamber. In addition, the gas injection part includes a plasma injection pipe 4 arranged in the plasma torch 3.

Here in this embodiment, for safe operation of the apparatus, parts other than the microwave power source 1 of the micro plasma source are incorporated in a draft chamber 7.

According to the preferred embodiment, the plasma torch 3 includes a cooling unit for cooling down the gas of temperature nonequilibrium condition before the gas is introduced into the plasma injection pipe 4. It is preferable that the plasma torch 3 includes a flow rate regulating valve arranged at a gas supply port of the chamber. Furthermore, it is preferable that at least one of the chamber of the plasma torch 3 or the plasma injection pipe 4 is provided with means for mixing steam in the gas of temperature nonequilibrium condition.

Thus, power is supplied from the microwave power source 1 to the plasma torch 3 through the co-axial cable 2. Gas is supplied from the gas cylinder 8 to the plasma torch 3. Then, plasma generated by the plasma torch 3 is irradiated to a sample 5 fixed to the substrate 6 so as to carry out sterilization.

In order to verity sterilization effect of the above-mentioned low temperature dry disinfection device, experiments were conducted as follows:

Frequency of microwave was 2.45 GHz, power was between 300 and 400W. Argon, helium, and oxygen were used as gas and maximum flow rate of the gas was 20SLM. A mixed gas of those gases could be used.

Irradiation distance was appropriately adjusted between 70 mm and 150 mm in such a manner that temperature on the substrate where the sample 5 was set became 323K, 333K, 353K, and 383K, respectively. The heated argon gas was supplied by supplying the argon gas through a stainless pipe heated by an electric heater. Sterilization time was set to 10 minutes, 20 minutes, 30 minutes, and 40 minutes, respectively.

For comparison with the low temperature dry disinfection device of the present invention, sterilization by ultraviolet irradiation was carried out. Using a mercury ultraviolet lamp (UV lamp), ultraviolet was irradiated to a sample.

Temperature of the gas was measured by E-type thermocouple. Bioindicator (3M, Attest 290) and a sample of No. 1291 were used. The sample is a piece of paper coated with spores of Bacillus subtilis existing in a natural environment.

Sterilization effect was checked by inserting the processed sample 5 into the bioindicator. When the sample is determined to be negative (−) by the bioindicator, it is guaranteed that the spores of Bacillus subtilis were sterilized by at least a log reduction number of 5 (10⁻⁵). On the other hand, when the sample is determined to be positive (+), it means that the log reduction number is less than 5.

Condition of spores of Bacillus subtilis was photographed by use of real surface microscope VE-7800 (product of KEYENCE). FIGS. 2-5 are photographs of spores of Bacillus subtilis coated on the surface of the samples obtained in this experiment. In FIG. 2, the spores of Bacillus subtilis are not processed, in FIG. 3, the spores of Bacillus subtilis are processed by the argon plasma irradiation (353K), in FIG. 4, the spores of Bacillus subtilis are processed by the heated argon gas irradiation (353K), and in FIG. 5, the spores of Bacillus subtilis are processed by the UV irradiation. Each of the spores have a length of 1 to 2 μm and a shape of a cocoon. Distribution of the spores is not uniform and the spores concentrate between fibers of paper. Difference in condition among the spores for each process is not clearly found in the photographs but the ratio of the concentration or the size of the spores became smaller depending on the processes.

FIG. 6 is a graph showing difference in the sterilization ratio by sterilization temperature and sterilization methods. In FIG. 6, the sterilization ratio (%) is defined by (the number of sterilized samples)/(the total number of the samples)* 100, where the sterilized samples represent the samples determined to be negative (−) by the bioindicator.

Following facts were found by the experiment:
(1) As the sterilization temperature rises, the sterilization ratio rises.
(2) Comparing between the argon plasma irradiation and the heated argon gas irradiation, the argon plasma irradiation has higher sterilization ratio.

1: A dry sterilization method comprising the steps of:
generating gas of temperature nonequilibrium condition containing high-energy particles by exciting gas; and
spraying pathogenic microorganisms with the gas of temperature nonequilibrium condition so as to kill the pathogenic microorganisms.

2: A low-temperature dry disinfection device comprising:
a gas supply source;
a high-energy particle generator for exciting gas supplied from the gas supply source so as to generate gas of temperature nonequilibrium condition containing high-energy particles; and
a gas spraying unit for spraying external pathogenic microorganisms with the gas of temperature nonequlibrium condition generated by the high-energy particle generator.

3: The low-temperature dry disinfection device according to claim 2, wherein the high-energy particle generator further comprises:
a chamber for receiving the gas supplied from the gas supply source;
an electromagnetic field generation unit for providing the chamber with an electromagnetic field for exciting the gas in the chamber; and
a high-voltage power supply for supplying the electromagnetic field generation unit with power voltage, the gas spraying unit further comprising a gas spraying pipe connected to the chamber.

4: The low-temperature dry disinfection device according to claim 2, wherein the high-energy particle generator further comprises a cooling apparatus for cooling down the gas of temperature nonequilibrium condition before the gas is introduced into the gas spraying unit.

5: The low-temperature dry disinfection device according to claim 2, wherein the gas supply source supplies a single type of gas or a mixed gas of more than two types of gases.

6: The low-temperature dry disinfection device according to claim 2, wherein the high-energy particle generator further comprises a flow rate regulating valve arranged at a gas supply port for receiving gas from the gas supply source.

7: The low-temperature dry disinfection device according to claim 2, wherein at least one of the high-energy particle generator and the gas spraying unit is provided with means for mixing steam into the gas of temperature nonequilibrium condition.
